(19) United States
(12) Patent Application Publication
(10) Pub. No.: US 2021/0186685 A1
(43) Pub. Date: Jun. 24, 2021

(54) SINGLE CIRCULAR CONVEX MAGNET LEAFLET DISC WITH AN OPPOSING UPPER AND LOWER MAGNETIC FIELD AND ELECTRONIC SEMICONDUCTOR SENSOR PROSTHETIC HEART VALVE THAT CAN COMMUNICATE FROM THE HEART TO THE BRAIN

(71) Applicants: Kyle Au, Logan, UT (US); Syltianna Au Au, Logan, UT (US)

(72) Inventors: Kyle Au, Logan, UT (US); Syltianna Au Au, Logan, UT (US)

(21) Appl. No.: 16/996,752

(22) Filed: Aug. 18, 2020

Related U.S. Application Data
(60) Provisional application No. 62/950,208, filed on Dec. 19, 2019.

(51) Int. Cl. A61F 2/24

(52) U.S. Cl. CPC A61F 2/246; A61F 2/2409; A61M 2205/50; A61F 2210/009

(57) ABSTRACT

A single circular convex magnet leaflet prosthetic heart valve with an opposing upper and lower magnetic field mounted into a C-clamp, with a modulating sensory unit within the top housing frame comprising; 1) a single, disc-shaped magnetic leaflet that opens and closes against an opposing magnetic main housing frame, designed to reduce friction in order to avoid hemolysis and coagulation. 2) an attached sensory unit that communicates from the heart to the brain through the braingate and microprocessor, compensating for the native defective heart valve. These sensors are regulated through brain guidance that might be induced by the sympathetic or parasympathetic system assisting with the vagus nerve to help tranquilize blood flow. This can replace the tricuspid or bicuspid valve. 3) a C-clamp used to prevent installation errors. Once the C-clamp is installed, un-installing and re-installing the entire unit will be unnecessary, thus, expediting the process, which could be lifesaving.

| | | |
|---|---|---|
| U.S. Pat. No. 4,661,106 | Apr. 28, 1987 | Moll |
| U.S. Pat. No. 3,824,629 | July 1974 | Shiley |
| U.S. Pat. No. 6,228,112 B1 | May 08, 2001 | Klootz et. Al. |
| US 2019/0314150 A1 | Oct. 17, 2019 | Forsell |
| U.S. Pat. No. 6,007,577 | Dec. 28, 1999 | Vanney |
| U.S. Pat. No. 5,215,088 | Jun. 01, 1993 | Normann et at. |
| US 2013/0046148 A1 | Feb. 21, 2013 | Tathireddy et al. |
| US 2019/0083800 A1 | Mar. 21, 2019 | Yang et at. |
| US 2018/0117324 A1 | May 03, 2018 | Schilling et al. |
| U.S. Pat. No. 6,045,576 | Apr. 04, 2000 | Starr et al. |
| US 2018/0154154 A1 | Jun. 07, 2018 | Sheldon et al. |

### CROSS-REFERENCE TO RELATED APPLICATIONS

This is the continuation of the application previously filed on Dec. 18, 2019, U.S. Provisional No. 62/950,208, and Oct. 30, 2019, U.S. Provisional No. 62/928,032, Magnetic Opposing Artificial Heart Valve which expands and to expressly claim and to apply and set the validity of utility patent.

### TECHNICAL FIELD

The present invention relates to a single circular convex magnet leaflet with an opposing upper and lower magnetic field and electronic semiconductor sensor artificial heart valve. More specifically, the present invention relates to a prosthetic single magnetic leaflet disc opposing magnetic main housing frame and electronic semiconductor sensory artificial heart valve for medical use to provide the input and response of the heart to the brain and receive signals from the brain to the heart. This magnetic opposing with sensory can be used for replacing the native bicuspid valve or tricuspid valve of the human heart.

### BACKGROUND OF THE INVENTION

Currently the artificial heart valve is divided into two kinds. First, the bioprosthetic heart valve that uses animal tissue such as bovine tissue, Porcine tissue or homograft tissue. Second, is the mechanical prosthetic heart valve such as Caged-Ball, Starr-Edwards et al. Tilting-Disc, Bjork-Shirley et. al, and the most successful bi-leaflet mechanical artificial heart valve from St. Jude. They are all built to help people to overcome a defective native heart valve ultimately designed to save lives.

However, the bioprosthetic heart valve is highly susceptible to deterioration caused by the patient's own immune system, because the immune system is designed to attack all foreign substances. Therefore, sometimes the bioprosthetic heart valve can be destroyed by bacteria that, invades patients' bodies because of the lack of protection from their own immune system. Likewise, the mechanical artificial heart valve will have a high risk of thrombogenicity and homolysis; therefore, a patient must take lifelong medication. Moreover, replacing the artificial heart valve from bioprosthetic valve or prosthetic valve is only solving temporary mechanical issue of the heart but the, blood clot, the feedback from chordate tendineae or SA node as well as future know of specific myocardial tissue would not be connected, response and cooperate with brain for long term problem.

Accordingly, there is a need for an artificial heart valve that will not rupture the blood cells and that will communicate with the brain and that respond to natural emotion such as respond to the SA node, the AV node, the pacemaker and the sympathetic and parasympathetic nervous system.

### FIELD OF THE INVENTION

It is a single circular convex magnet leaflet with an opposing magnet and semiconductor sensor prosthetic human heart valve that helps pumps blood in one-way direction acting more like the function of the natural human heart; because this invention is designed to improve their quality of life by interconnection of heart to brain communication, furthermore, minimize friction and reduce medication intake.

### SUMMARY OF INVENTION

The single circular convex magnet leaflet prosthetic heart valve has opposing upper and lower magnetic fields with a semiconductor sensory that aims to solve hemolysis and coagulation problems from a heart valve transplant. This invention uses a single convex leaflet magnet valve. The convex shape helps fluid flow down hydrodynamically and is constructed concave disc that helps fluid flow smoothly. The main housing frame can be constructed of magnetic or nonmagnetic material. If the main housing frame is made of nonmagnetic material then adding an upper center magnet disc to oppose the single circular convex leaflet valve helps reduce the hard impact of single leaflet valve to the main housing frame, result lessening the number of shattered blood cells. The main circular shaft single hole guides the single circular convex magnet leaflet disc that slides up and down producing a soft friction. Adding the rear magnet to the rear of the shaft and helps stabilize the upright position of valve preventing an unexpected blackout when a patient moves or stands up. This artificial heart valve has an electronic semiconductor sensor that sends signals to the brain. Sensor might connect to the SA node, the AV node, the vagus nerve and/or the sympathetic and parasympathetic nervous system. The rear end of the main central shaft has two coils. Coil 1 will receive signals from braingate that processes from the semiconductor sensor and coil 1 pulls the single circular convex magnet leaflet disc down to open and lets blood flow into the other chamber or pushing the concave disc up to close. Its main function is to control bradycardia and tachycardia. Coil 2 is located in the inner rear end center of the opposing rear magnet disc. Coil 2 job is to receive signals from brain to hold the valve open a little longer after coil 1 has initiated its signal. It is control by the computer module, braingate, and microprocessor. Coil 2 is further guaranteeing to control of tachycardia as well as, bradycardia.

Last, let's look at the C-clamp. The biofabric cuff is wrapped around the C-clamp making it easy for surgeons to attach it to a patients heart tissue (U.S. Pat. No. 6,007,577 Vanney et. al. and U.S. Pat. No. 6,045,576 Starr et al.) The surgeon first secures and installs the C-clamp to a patient's heart tissue regardless of the direction of blood flow. This main critical C-clamp FIG. 4, 100 can be enlarge C-clamp diameter. This make is possible for cardiologists to easily exchange or flip the prosthetic artificial heart valve (up to down or down to up) if needed to correct the direction of the blood flow.

This will eliminate most errors so surgeons won't have to uninstall and reinstall the entire unit. This decrease of redoing the artificial heart valve will save time and could mean the difference between life and death to many patients in the operation room.

### BRIEF DESCRIPTION OF THE DRAWINGS

To further clarify the above drawings, FIG. 12A-12B the main housing frame embodiment 20 is a unit, while 114a and 114b is the axially magnetized field. Likewise, FIG. 13 the single circular convex leaflet top view 14a is a unit while 19 is the edge of the 14a embodiment divided into 19a and 19b is the axial magnetized field. The objects shown in the figures can be compared to current prosthetic artificial heart valves. Clearly the description can be expanded more to understand the single circular convex leaflet magnet opposing magnet functions shown in FIG. 14C the real concept characters refer to all parts throughout magnet opposing magnet:

FIG. 1 The top view surface looking into the single circular convex magnet leaflet disc magnetic opposing the magnet and include C-damp sensory.

FIG. 2 Images of prosthetic heart valve housing with C-clamp, showing from the rear images of the magnet opposing magnet and sensory artificial mechanical heart valve.

FIG. 3 A perspective has three prosthetic artificial magnet opposing magnet heart valves implanted to human heart. The aorta heart valve is in behind so it is not shown. Arrow is indicating of blood flowing.

FIG. 4 Illustrates a complete standing upright position of the single circular convex magnet leaflet artificial heart valve with C-clamp on it.

FIG. 5 Shows a rear view, slightly tilted, of FIG. 2 to show the three dimensions and a more clarified side view.

FIG. 6 Illustrates a standing upright position of a barebone housing frame of this artificial heart valve with no c-clamp, no shaft and no single magnet leaflet disc on it.

FIG. 7 Shows a quarter view that how the c-clamp fits into groove of the main housing frame.

FIG. 8A Illustrates a cutting view showing the entire right half of the prosthetic artificial heart valve. The valve is in closing position.

FIG. 8B Is a cutting view showing the entire right half of the prosthetic artificial heart valve. The valve is in opening position.

FIG. 9 Deployed position cutting view of the entire right half of the artificial heart valve with 2 semiconductor sensors inside of 23 on top, 2 magnet coils sensor on bottom.

FIG. 10 Cutting view of the entire right half of the magnetic opposing magnet artificial heart valve disc, showing 19 magnet opposing 18 magnet.

FIG. 11 Cutting view of the entire right half of the artificial heart valve. It shows the total assembly of the single circular convex magnet opposing magnetic concave rear view disc, the sensors, the shaft, the magnet coils etc.

FIG. 12A-12B Shows the cutting haft of single convex leaflet magnet and opposing magnet on the magnet housing frame. Shows the upper ring 114 proximal inflow-end diameter is slightly smaller than the lower ring 115 distal outflow-end diameter.

FIG. 13 Cut view of the single circular convex 14a magnet disc upper view and concave 14b bottom view.

FIG. 14A The complete circle 14a convex leaflet magnetic disc upper view while 14b concave in the bottom view. 14 is the hole.

FIG. 14B Shows the simple axially magnetized magnet vs diametrically magnetized magnet. N and S are nonspecific relating to the invention of a single circular leaflet magnet disc. It is just for demo of axially different than diametrically magnet.

FIG. 14C Shows the natural force of the magnet is that like, poles repel like poles.

FIG. 15 Shows C-clamp that fit into the main housing frame of the prosthetic artificial heart valve.

FIG. 16 the main central supporting shaft and in the bottom might or might not have magnet coil. It depends on medical need.

FIG. 17 Shows the entire single circular convex magnet leaflet disc opposing magnet housing frame and included C-clamp.

FIG. 18 Illustrates the operation of this magnet opposing magnet with sensory artificial heart valve and computer module in human heart and/or artificial heart (provisional 62/928,032 Oct. 30, 2019 Kyle Au.)

FIG. 19 Pictures showing the image braingate “Cyberkinesis Micro Array” #200 Richard Normann et al. U.S. Pat. No. 5,215,088, Jun. 1, 1993

### DETAIL DESCRIPTION OF THE PREFERRED EMBODIMENT

When people have a defective heart valve, it can be successfully replaced by a bioprosthetic or prosthetic mechanical heart valve. The bioprosthetic heart valve is a valve that is made from porcine tissue, bovine tissue, or homograft tissue. The prosthetic heart valve is a valve that is made from nonliving materials such as stainless steel, titanium, pyrolytic carbon, etc. After an artificial heart valve replacement, all patients must take medications to prevent hemoglobin toxicity because blood cells will be lysis by the clapping and collision of the valve to the housing frame causing hemolysis. Normally, clotting factor proteins are everywhere in our bodies and will protect us from cuts and injuries. In general, all external drugs are toxic. When taking any kind of drug long term such as Warfarin, Eliquis, Nitropress, Dobutrex, or lnocor, etc, either an unmetabolized accumulation of the drugs or metabolized waste of the drugs may remain in a patient's body. When the blood cells are damaged, their internal substances (enzymes, proteins, lipids, irons, hemoglobin, metalloproteins, and clotting factor in plasma) leak out and become toxic. The body will react and activate it natural clotting factors and increase the risk of stroke. It can even damage the lungs and liver as well as the kidneys. Warfarin, Heparin, and Eliquis are blood thinners and their toxicity and side effects are unavoidable. Patients also must carefully control the intake of certain green vegetables such as broccoli, kale, spinach, and others that might react to the medications. Research speculates that after an artificial heart valve replacement, patients lose control of certain involuntary heart signals to the brain and feedback from the brain to heart. Consequently, a patient's daily activity is constrained and the quality of life is lessened day by day.

My invention, the Single Circular Convex Magnet Leaflet Opposing Magnet with the Semiconductor Sensors and Coils Prosthetic Heart Valve might help patients enjoy a better quality of life. The benefit of my heart valve magnet opposing magnet invention reduces the risk of hemolysis and also provides feedback from the sympathetic and parasympathetic nervous system from the brain to the braingate, which then processes in the microprocessor, and thenceforth, regulates the heart beats through coil 1 and, coil 2, further reducing drug intake.

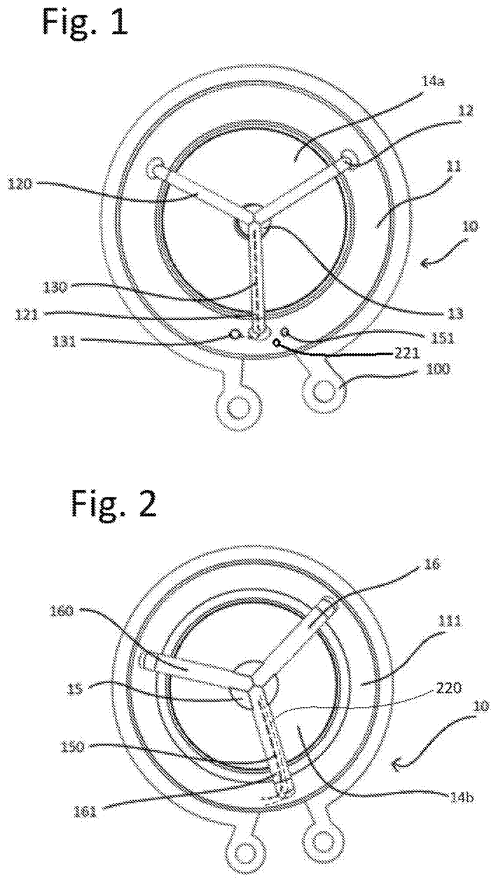

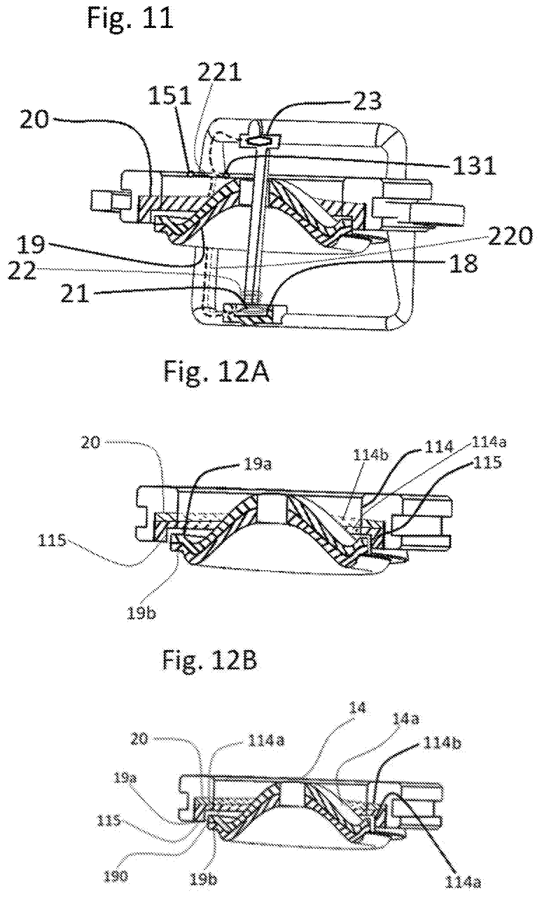

FIG. 1 This is the top view. It shows the layout parts of the prosthetic single convex magnet leaflet opposing magnet, annulus housing frame. The valve is in closed position. This maintains a one-way flow of fluid. 10 is C-clamp that can be made with an enclosed biofabric suture ring like U.S. Pat. No. 6,045,576 Starr et al., and is sewn into the heart first. This C-clamp is wrapped with biofabric and later hold the main housing frame in place. The view of C-clamp 10 embraces the prosthetic artificial heart valve. 100 are two nonspecific holes on the C-damp that can be widened with a tool to enlarge it diameter, unlocking the main housing frame, and allowing the surgeon to exchange or flip the prosthetic artificial heart valve as needed. The natural, unexpended C-clamp diameter position will automatically hold and lock the main housing prosthetic heart valve in place. The benefit is that the cardiologist won't have to un-install or re-install the entire artificial heart valve unit because of mistaking the direction of blood flow, thus making it easier to switch and replace a prosthetic heart valve, saving time and the saving lives of patients. 11 is the upper view of the main housing frame and can be made of a magnetic or a nonmagnetic circular inflow. This inner diameter is slightly smaller than that of the outer main housing frame showed in FIG. 11, and FIG. 12. The inner inflow-end proximal diameter of 20 unit is 114 and the outer outflow-end distal diameter is 115. The three upper mandrels 12, 120 and 121 are connected to the center semiconductor sensor disc 13 and secures the structure of heart valve. The 13 can just be an electronic semiconductor sensor or semiconductor sensor with an outer magnetic disc. 14a is the single circular convex leaflet magnet disc valve with the center hole connecting to the main shaft 17 (the main shaft 17 is not show in FIG. 1). 14a is the single circular convex leaflet magnet that helps the hydrodynamic flow of blood press down and past disc 14a, thus, the blood will flow into the chamber below. Images 131, 151, and 221 are the three outlet plugs from the main magnet housing frame that connect, from the semiconductor sensor or magnet coils to the braingate. 130 is the impregnated wire in the mandrel of 121.

FIG. 2 Is the rear view of the prosthetic heart valve housing with the 10 C-damp embracing the single circular convex magnet opposing magnet with semiconductor sensor. 14b shows the single circular concave magnet leaflet and this is the rear view of 14a looking into the magnet opposing magnet which shows a one-way flow of fluid in its closed position. The purpose of the concave leaflet is to help the hydrodynamic flow of the systolic pressure, while high pressure from the rear concave valve pushes the leaflet up into closed position. 111 is the rear of the main magnetic or nonmagnetic housing frame. The inner orifice diameter 114 is slightly larger than the inner diameter of the outer orifice 115 so valve 14a can slide down to open the valve, this relate to FIGS. 12A and 12B of cut side view. 15 is the outer magnetic and inner coil disc in the rear end that holds and secure the three mandrels together. 16, 160, and 161 are the three mandrels that are attached from the main housing frame extending down to secure the prosthetic heart valve. 150, and 220 indicate the impregnated wires in the 161 mandrels at the rear housing frame.

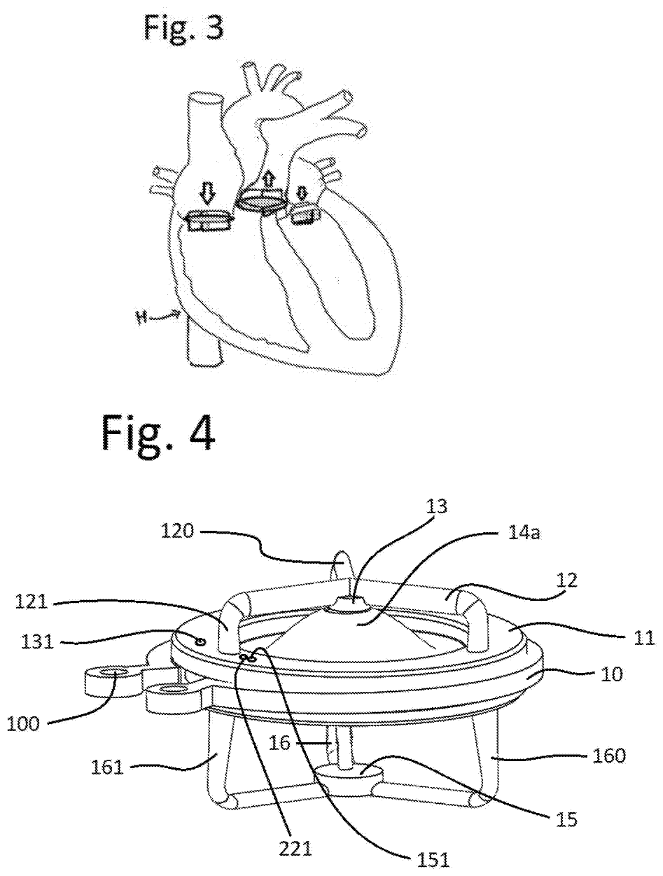

FIG. 3 Is a picture of the heart that has three prosthetic magnet opposing magnet and sensory artificial mechanical heart valves. FIG. 3 shows that the aorta valve is behind so it is not easy to see the implanted prosthetic artificial heart valve. The arrows indicate the direction of the blood flow.

FIG. 4 Is showing an upright position of the single convex magnet leaflet opposing magnet of the main housing frame and sensor heart valve with a general overview of all parts with the C-clamp in place. The image of the prosthetic valve has a single convex magnetic disc. The single circular convex magnetic disc can, slide down to open and let blood flow down and out into the different chambers. 10 is the C-clamp. 11 is the upper main magnet housing frame. 12, 120, and 121 are the three mandrels extending from the main housing frame to unite with 13 the magnet with an electronic semiconductor sensor or just semiconductor sensors disc, 14a is the single circular convex magnet leaflet disc. 131, 151, and 221 are the connection outlet sensor plugs that lead to the braingate. 16, 160 and 161 are the lower mandrels that extend from main housing frame and hold the prosthetic heart valve secure in the rear part. 15 is the rear magnet and inner contain 21 (not shows in FIG. 4) is the inner coil 2, this coil helps prevent bradycardia and tachycardia by sending signals to the braingate.

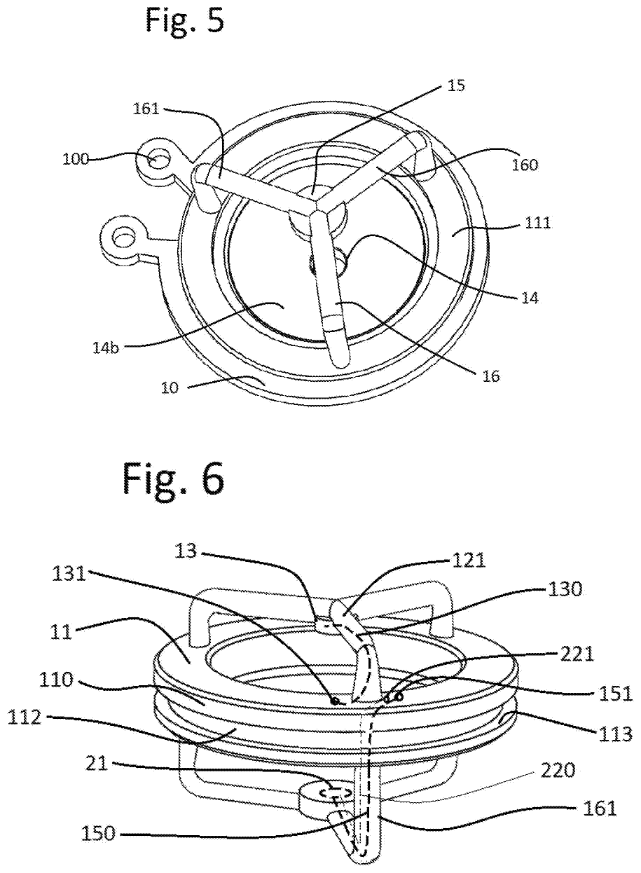

FIG. 5 is the rear view of the upper top of the prosthetic single circular convex magnet leaflet and is shown slightly tilted of FIG. 2. the singular circular concave magnet leaflet is 14b; concave in the rear view. 14 shows the center hole of the singular circular magnet leaflet disc; concave in the upper view. Blood can only flow out.

FIG. 6 Shows the general upright position of the main frame with no C-clamp, no single leaflet disc, and no main shaft. It does, however, show that 130 is impregnated inside 121 and this is one of the upper three mandrels, and 130 is connected to 131, the electronic semiconductor sensor plug outlet. Likewise, wire 150 is impregnated inside 161 and this is one of the rear three mandrels connected to 151, the outlet coil plug and 21 is coil 2. It shows wire 220 is impregnated inside 161 and is one of the rear three mandrels connected to 221, the outlet coil plug and 22 is coil 1. Coil 1 is inside of the rear main circular main shaft 17 (this main shaft and coil 1 are not show in FIG. 6).

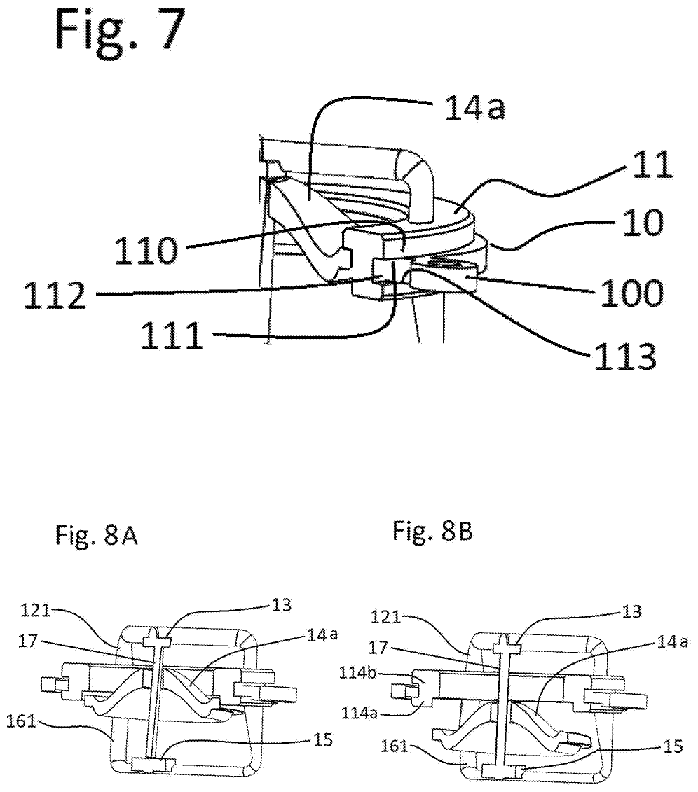

FIG. 7 Shows the outer frame housing of the artificial mechanical heart valve cut to a quarter portion. The inner frame 10 of the C-clamp is held tight to fit the outer ring of 112. As indicated, 11 is the upper frame of the single circular convex leaflet magnet opposing magnet housing main frame. 110 is the upper outer and lateral annulus ring. 113 is the bottom lateral inside while 111 is the inner upper side of the artificial housing frame. 111 and 113 fit snugly into the C-clamp indicated 10 and it is the bottom portion of 11. The C-clamp, in position, will hold the prosthetic magnet opposing magnet heart valve in place. The C-clamp is un-expanded and in locked position. This C-clamp holds the prosthetic artificial heart valve in place.

FIG. 8A Shows the prosthetic single circular convex leaflet magnet opposing magnet with semiconductor sensor heart valve cut in half and views into the internal structure. Convex valve 14a slides up, and is shown in closed position.

FIG. 8B Shows the artificial valve cut in half and views into the internal structure. Valve 14b is the bottom view of the concave disc showing the valve in open position.

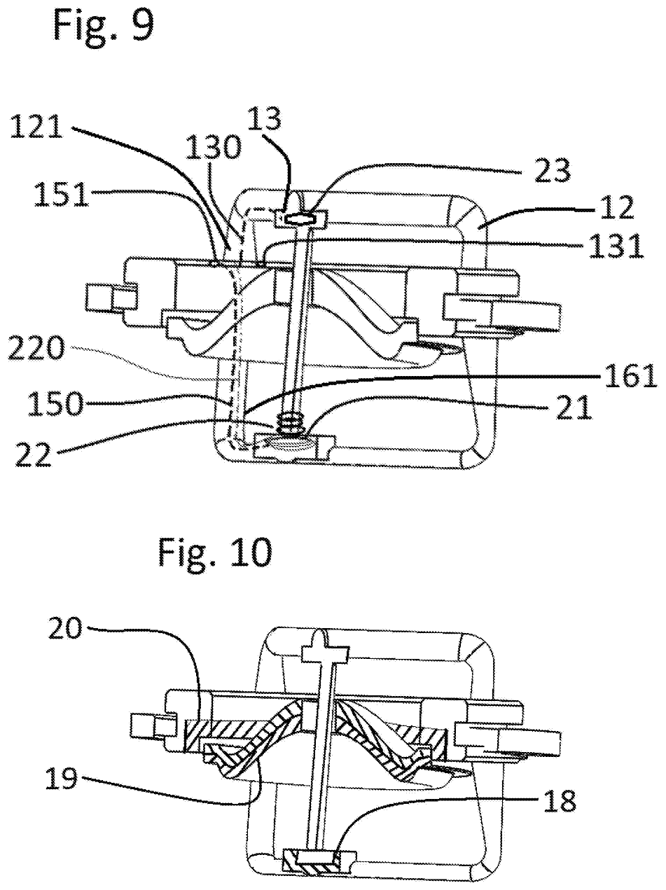

FIG. 9 Shows the artificial valve cut in half with, a view of the internal structure. The internal wire indicated 130 is impregnated inside of the upper mandrel of 121. 23 has two electronic semiconductor sensors and 23 is inside 13. The inner coil 22 that wraps around the bottom of 17 shaft that help to control bradycardia and tachycardia. The exterior of 13 whether can be made out of either the semiconductor sensors or semiconductor sensors with an outer magnet washer disc.

FIG. 10 Shows the artificial valve cut in half with a view of the internal structure. This is the main frame of magnet 20 opposing from 19a the single circular convex magnet leaflet disc. 19b is opposing to 18, the magnet in the rear. Notice that if the housing main frame 20 is nonmagnetic then 13 must be magnetic to create the opposing force to 19a at the top, either to 13 (or to the three top mandrels might be magnet) for that magnet opposing magnet effect to avoid hemolysis.

FIG. 11 Combines FIG. 8-10. This acts as magnet opposing magnet sensory unit that sends signals of heart beats from the heart to the braingate, and the braingate might send signals to the microprocessor and the computer module that is connected to the braingate as well as the SA node or the AV node and pacemaker to regulate heartbeat.

FIG. 12A Shows the magnet annulus ring main housing frame proximal diameter of 114 is a little smaller than distal 115 diameter so that the valve can slides up to close or slides down to open. 20 is a unit while divided to 114a and 114b magnetically field. The magnet annulus ring housing main frame axial magnetized of 114a opposes to 19a magnetic field of the single circular leaflet magnetic field.

FIG. 12B. 190 shows axially magnetized of 19a and 19b and 190 is the edge of 14a and 14b a single circular magnet leaflet disc. FIG. 12B shows the gap between 115 and 190 is very small.

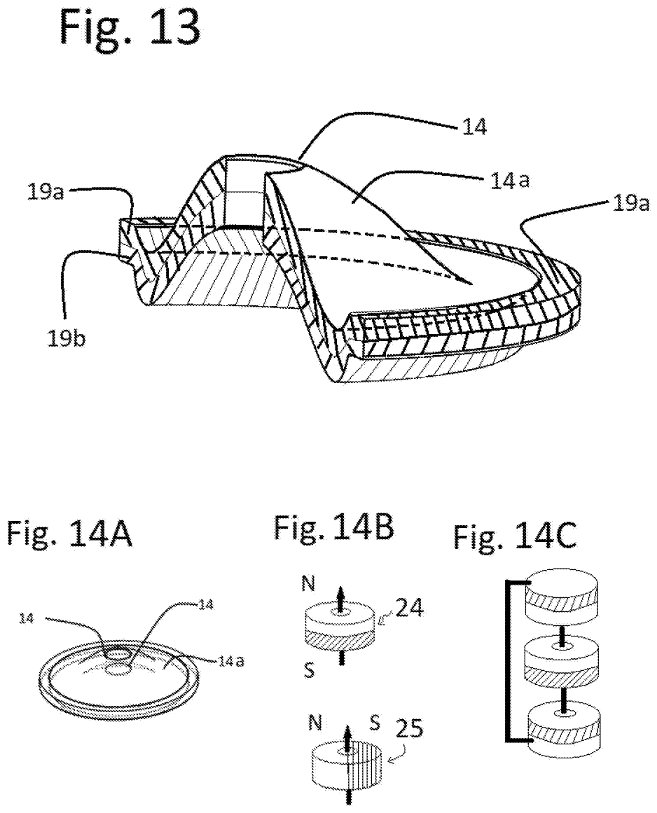

FIG. 13 The convex disc 14a cut section shows the upper magnet is convex while the lower is concave creating a fluid dynamic that strengthens the hardness of the disc. This is indicated by 19a and 19b showing the disc as being axially magnetized; it is either north pole to south pole or south pole to north pole. This will have the opposing affect at the top of the housing and it is arranged to oppose the lower magnet of 18 as well as the opposing magnet on the top of 20

FIG. 14A Shows the complete circular upper disc 14a, with the central hole 14.

FIG. 14B Magnet 24 shows an axially magnetized field vs magnet 25 which show a diametrically magnetized field.

FIG. 14C Shows the magnet conception of like pole opposing like pole (show N or S is nonspecific).

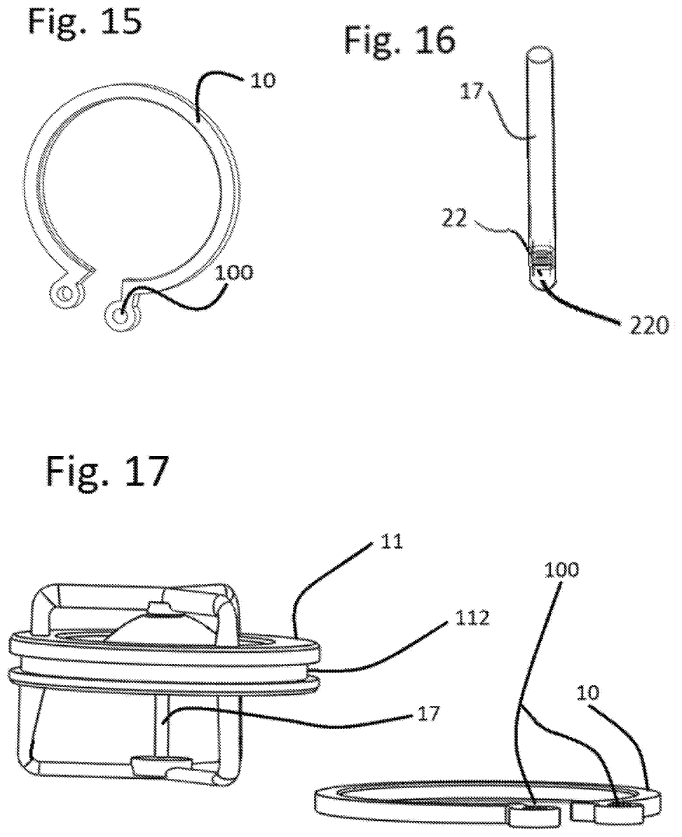

FIG. 15 Shows the C-clamp. This can combine with the prior art of Vanney et al biofabric cusp to be sewn into the heart first, either the aorta, the mitral valve, or the pulmonic valve. This prosthetic single circular convex leaflet magnet opposing magnet can replace a tricuspid or a bicuspid valve.

FIG. 16. Shaft 17 image is the assembly of the main circular shaft to be connected into the main central artificial heart valve housing. In the rear end there is coil 1 indicated as 22. It is in the inner of shaft. 22 is connected to the wire 220 inside the 161 mandrel and leads to outlet plug 221, which send signals to the braingate.

FIG. 17 Shows the complete image of the single circular leaflet magnet opposing, magnet with 10 C-clamp and is ready for installation.

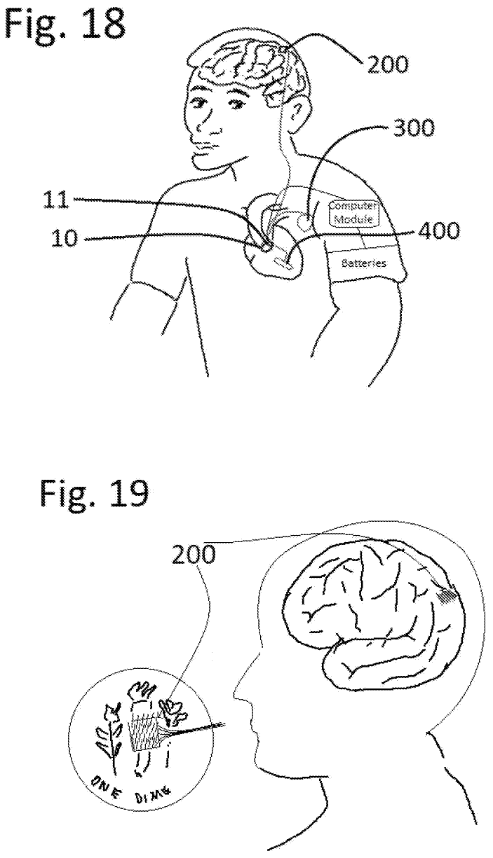

FIG. 18 Shows the attachment of the magnetic opposing heart valve communicating with the pacemaker 300, nonspecific. 400 is the newer Metronic pacemaker patented by Micra. It is also receiving and communicating with the braingate and is controlled by a computer module that regulates the heartbeat.

This invention claimed is:

1. A single circular convex magnet leaflet prosthetic heart valve with an opposing upper and lower magnet and an electronic semiconductor sensory unit within the artificial heart valve housing that can communicate from the heart to the brain and regulates blood flow, comprising;
a single disc-shaped leaflet heart valve having a circular convex washer and having an axially magnetized orientation also having a center hole and,
a main single housing frame that has an annulus ring that can be either magnetic or nonmagnetic that said;
hereto, a central single circular main shaft guides a single circular magnetic leaflet disc valve sliding up to close and down to open, and is also connected to the upper semiconductor sensor, the rear inner magnetic coil 1 inside shaft, and the rear end with a small washer magnet disc, and the small washer magnet disc inside has coil 2 said activator that has,
the connection of three equally distributed mandrels at the mid-upper perimeter of the main housing frame that has a semiconductor sensor disc and said,
one of the three equal distributed mandrels at the mid-upper perimeter of the main housing frame extending up which has an impregnated wire; furthermore,
one of the three equally distributed mandrels at the mid-rear perimeter of the main housing frame extends down and has two impregnated wires, and said,
structurally includes a pyrolytic carbon magnet or Neodymium magnet as well as Samarium Cobalt, and;
wherein, at least one layer comprises zirconium oxide or metal oxide polymer,
an outermost layer fits into the annuloplasty C-clamp and embraces the prosthetic magnetic opposing magnetic ring said, there is a larger ring that has a groove to fit the C-clamp.

2. A single disc-shaped leaflet heart valve having a circular convex washer of claim 1, the disc with a single circular convex washer top view that helps hydrodynamic fluid flow at diastolic pressure and valve open, and wherein, when the concave rear view for hydrodynamic fluid at systolic pressure and valve close.

3. A single disc-shaped leaflet heart valve having a circular convex washer of claim 1, and claim 2, constructively, the single circular convex magnet leaflet heart valve disc washer is constructed of an axial magnetized orientation, meaning that the upper surface of leaflet is constructed of either the N or S magnetic field, and lower surface that is the opposite magnetic field.

4. A single disc-shaped leaflet heart valve having a circular convex washer of claim 1-3 the washer has a center hole for the center of the center shaft of the prosthetic heart valve that guides the leaflet disc as it slides down to open the valve and shows the single circular convex magnet leaflet disc that slides up to close the valve.

5. A main single housing frame that has an annulus ring of the magnet; claim 1, that interacts with claim 3, shows the main housing frame annulus ring with a proximal ring inflow-end that is slightly smaller in diameter than the distal ring outflow-end diameter.

6. A main single housing frame that has an annulus ring of the magnet; claim 1, that interacts with claim 3, 5, shows the main housing frame annulus ring that can be made of an axially magnetized magnet with a magnetic field that opposes the upper surface of the single circular convex magnet leaflet valve, which is a circular convex magnetic disc, characteristic of this opposing magnet, forcing the leaflet down against the housing frame, which reduces the breakdown of blood cells and prevents hemolysis and unwanted coagulation of blood.

7. A main single housing frame that has an annulus ring of a nonmagnet; claim 1, that interacts with claim 3, and is similar to claim 5; the main single housing frame can be constructed with a nonmagnetic annulus housing ring; however, if the annulus ring of the main housing frame is constructed with a nonmagnetic housing frame annulus ring then a small upper centrally axially magnetized disc magnet must be attached in the center at the top of the upper center housing frame, resulting in the same effect as having a magnetic annulus ring opposing magnetic housing to the single circular convex magnet leaflet valve, reducing the main impact of the leaflet valve against the housing frame, which reduces the rupture of blood cells and prevents hemolysis and unwanted coagulation.

8. A central single circular main shaft guides a single circular magnetic leaflet of claim 1, interacting with claim 4, 5, the main shaft helps guide the single circular convex magnet leaflet valve slide downward to open and slide upward to close valve.

9. A central single circular main shaft also connected to the upper semiconductor of claim 1, the shaft's upper end is connected to an electronic semiconductor sensor disc in the center top of the unit and said, this sensor detects the heart beat and sends signals to the microprocessor that connects to the SA node or vagus nerve, as well as the pacemaker that interact with the sympathetic and parasympathetic nervous system that help regulates our emotions such as sympathetic and parasympathetic and affects blood flow based on feedback from the brain to the SA node and the AV node and this will be expand and explain in the next utility artificial heart (patent number 62/928,032, filed Oct. 30, 2019 Kyle Au.)

10. A central single circular main shaft connected the rear inner magnetic coil inside the main shaft of claim 1, claim 8, the shaft at the rear end and that is connected to the magnetic disc and this inner shaft has a coil 1 to control a single leaflet heart valve moving down to open and smooth blood flow, said this coil 1 is connected to the braingate and the main function is to regulate unwanted bradycardia and tachycardia.

11. A central single circular main shaft connected to the rear small magnetic washer disc of claim 1, the shaft in the rear end is connected to an axially magnetized magnet washer disc that holds, stabilizes, and guides the single circular convex magnet leaflet valve up and the affect is the rear opposing magnetic field holds the single leaflet in the high upright position, said this rear small magnet washer disc and the single circular concave magnet leaflet are opposing and repel each other henceforth, this helps patients avoid blackouts when they suddenly stand up, and also helps blood flow smoothly even when a patient is lying down.

12. A central single circular main shaft connected to the rear small magnet washer has a coil 2; coil 2 to clarify the configuration and said coil 1 described in claim 10, is in the rear bottom inner of the shaft, and when initiated it pulls the magnetic leaflet disc down to open the valve resulting in smoother blood flow, and about this coil 2; which holds the single circular concave magnet leaflet valve down to keep the valve open a little longer or magnetic pushes up faster and might be controlled by the vagus nerve or pacemaker keeping the rhythm of the heartbeat, and responds to signals from the brain to the braingate (Normann et al) and then to the microprocessor and said the coil 2 function is to reinforce from coil 1 and to guarantee the control of avoiding bradycardia and tachycardia.

13. The connection of three equally distributed mandrels is connected at the mid-upper perimeter of the main housing frame claim 1, and has an impregnated second sensor secured disc to the main upper shaft has a semiconductor sensor connecting to the braingate, then it can be analyzed by the microcomputer processor with the function to avoid fibrillation.

14. Claim 9 and claim 13 are two sensors in the same upper disc, moreover, this disc connected the three mandrels and secure the upper prosthetic heart valve, more specifically claim 9 for bradycardia and tachycardia and claim 13 are worked counter other for defibrillation and control the irregular of the heartbeat.

15. One of the three equally distributed mandrels at the mid-lower perimeter of main housing frame; claim 1, has two impregnated wires inside, and the first is from the magnetic coil 1 from the inner shaft, and the second wire is from the magnetic coil 2 in the lowest inner magnetic disc. They carry the signal to the sensor, which carries the signal to the braingate (Richard Normann et al), then it will be analyzed in the microcomputer processor and communicate with the brain.

16. Structurally includes in claim 1, the construction of the single circular convex magnet leaflet washer and the main housing frame, materials comprise of a Pyrolytic Carbon Magnet, Neodymium or Samarium Cobalt.

17. At least one layer comprises; the prosthetic heart valve of claim 1 wherein, at least one layer comprises a zirconium oxide and/or metal oxide.

18. An outermost layer that fits into the annuloplasty C-clamp of claim 1, the outermost layer fits into the annuloplasty C-clamp that attaches to the outer most frame of claim 5, 6, and said the job of this C-clamp is to embrace the prosthetic magnetic opposing magnetic ring and is said to make it easier for the surgeon to focus on the main operation, and instead of worrying about the inflow or outflow of blood, cardiologist can concentrate on securing the C-clamp annuloplasty to the heart tissue and other necessities to avoid cerebral or myocardium infarction, and since installation of the C-clamp is fast and easy, surgeons will not have to un-install and re-install the entire unit because of the misdirection of blood flow, thus, the faster replacement of an artificial valve saves time and saves lives.
